(19) United States
(12) Patent Application Publication
(10) Pub. No.: US 2024/0386988 A1
(43) Pub. Date: Nov. 21, 2024

(54) FRAMEWORK TO TEST LEARNING OF NEURONAL CELL CULTURES

(71) Applicant: MICROSOFT TECHNOLOGY LICENSING, LLC, Redmond, WA (US)

(72) Inventors: Weiwei YANG, Seattle, WA (US); Benjamin Franklin CUTLER, Seattle, WA (US); Christopher Miles WHITE, Redmond, WA (US); Whitney HUDSON, Woodinville, WA (US)

(21) Appl. No.: 18/382,010

(22) Filed: Oct. 19, 2023

Related U.S. Application Data
(60) Provisional application No. 63/503,400, filed on May 19, 2023.
(60) Provisional application No. 63/503,406, filed on May 19, 2023.
(60) Provisional application No. 63/503,655, filed on May 22, 2023.

(51) Int. Cl. G16B 5/00; C12N 5/0793

(52) U.S. Cl. CPC G16B 5/00; C12N 5/0619

(57) ABSTRACT

This disclosure provides techniques and systems for evaluating the performance and learning of neuronal cell cultures. This is done with a collection of reference environments that provide reinforcement learning tasks. A neuronal cell culture is communicatively connected to a conventional electronic computing device that provides inputs and measures outputs. The connection may be implemented, for example, with a multi-electrode array (MEA). A reinforcement learning “gym” is created from multiple reinforcement learning tasks that can be provided to a neuronal cell culture in a standardized way. The neuronal cell cultures are trained to perform the tasks by use of rewards. This provides a standardized framework to evaluate biological learning in neuronal cell cultures so that different types of neuronal cell cultures can be compared. Standardized and reproducible techniques for evaluating the learning of neuronal cell cultures aids the development of neuronal cell cultures as compute substrates.

### PRIORITY APPLICATIONS

This application claims the benefit of and priority to U.S. Provisional Application No. 63/503,400, filed May 19, 2023; U.S. Provisional Application No. 63/503,406, filed May 19, 2023; and U.S. Provisional Application No. 63/503,655, filed May 22, 2023, the entire contents of which are incorporated herein by reference.

### BACKGROUND

The demand for computing power is increasing. For example, sophisticated and resource-intensive Large Language Models (LLMs) are now used by millions of people every day. Enormous amounts of computational resources are required to train and use modern artificial intelligence such as LLM's. However, the capacity of computing hardware is not keeping up with the increased demand. The monetary and energy costs required to train and operate ever more powerful systems to serve an increasing number of users could be a barrier to further development and adoption of artificial intelligence. Additionally, the projected future demand for computing resources will have significant energy and environmental costs unless more efficient computing systems are developed. Presently, there are not enough resources to provide ubiquitous and constant access to resource-intensive computing such as artificial intelligence.

To enable continued innovation in artificial intelligence and to support the anticipated increase in demand for computing power, alternative computing frameworks are necessary. Standardized techniques and systems for training and evaluating alternative computing frameworks can aid in their development. This disclosure is made with respect to these and other considerations.

### SUMMARY

Biological computation is a billion times more energy efficient than silicon computation. The adoption of biological analogs of machine learning—using actual neurons to create a neural network within a computer system—is an energy efficient way to implement artificial intelligence. To do so, a neuronal cell culture is coupled to a conventional electronic computing device. For example, neurons can be cultured on a multi-electrode array (MEA) that provides electrical stimulation to the cells and detects electrical activity of the cells. An interface couples the biological components to the electronic. The MEA is one example of an interface but there are other types of devices that can provide inputs to a neuronal cell culture and detect the neurons firing. There are also many possible types of neuronal cell cultures that can be used. One type is the cortical organoid or “mini brain” which is a three-dimensional structure formed from a cluster of neurons. The neurons can come from a variety of sources such as human or mouse cells.

This disclosure provides a framework to test the learning and performance of biological compute substrates that include neuronal cell cultures. A neuronal cell culture can be trained to perform computational tasks in conjunction with a conventional electronic computing device. There are multiple ways to create a compute framework from a neuronal cell culture as well as multiple ways to train a neuronal cell culture to perform specific computational tasks. To develop neuronal cell cultures into an effective framework for biological computing, techniques to understand how neuronal cell cultures learn are needed. Currently no such frameworks exist to evaluate biological learning of neuronal cell cultures as part of a computing device. This disclosure introduces a standardized framework that can be used to evaluate the performance of neuronal cell cultures on multiple different tasks such as a variety of machine learning tasks including specifically reinforcement learning tasks. Reinforcement learning is a type of machine learning where an agent learns to make decisions by interacting with an environment in a goal-direct manner and receiving feedback for its actions.

This framework is analogous to the reinforcement learning “gyms” used for evaluating conventional machine learning algorithms. Those conventional “gyms” allow a developer to test a machine learning algorithm on multiple different tasks in a collection of pre-built environments. A “gym” for neuronal cell cultures is based on standardized methods for interacting with cell cultures and the delivery of stimulus, such as through the use of an MEA, together with standardized reference environments. This provides a standardized framework to introduce relevant reinforcement signals, such as rewards, and to study how a particular neuronal cell culture design performs across multiple reinforcement learning tasks. For example, an Application Programming Interface (API) may be used to standardize how a neuronal cell culture interacts with a suite of different reinforcement learning tasks.

This standardization makes it convenient to test multiple different neuronal cell cultures on different combinations of reinforcement learning tasks. A given neuronal cell culture can be tested on multiple different reinforcement learning tasks or multiple different neuronal cell cultures can be tested on the same reinforcement learning task. Use of this type of “gym” framework rather than one-off testing makes it easier to perform a direct comparison of various techniques for creating and training neuronal cell cultures. It also makes it easier to compare the functionality and capability of different types of neuronal cell cultures. Researchers can focus on the development of neuronal cell cultures as compute substrates without needing to create custom simulation and testing environments. Use of neuronal cell cultures as compute substrates also makes it possible to observe how small changes in the environment of the neuronal cell cultures affects their computing ability. The small changes could be caused by exposing the neuronal cell cultures to a drug and observing how the drug affects the ability of the neuronal cell cultures to perform a reinforcement learning task.

Features and technical benefits other than those explicitly described above will be apparent from a reading of the following Detailed Description and a review of the associated drawings. This Summary is provided to introduce a selection of concepts in a simplified form that are further described below in the Detailed Description. This Summary is not intended to identify key or essential features of the claimed subject matter, nor is it intended to be used as an aid in determining the scope of the claimed subject matter. The term “techniques,” for instance, may refer to system(s), method(s), computer-readable instructions, module(s), algorithms, hardware logic, and/or operation(s) as permitted by the context described above and throughout the document.

### BRIEF DESCRIPTION OF THE DRAWINGS

The Detailed Description is described with reference to the accompanying figures. In the figures, the left-most digit(s) of a reference number identifies the figure in which the reference number first appears. The same reference numbers in different figures indicate similar or identical items. References made to individual items of a plurality of items can use a reference number with a letter of a sequence of letters to refer to each individual item. Generic references to the items may use the specific reference number without the sequence of letters.

FIG. 1 shows a neuronal cell culture connected to an electronic computing device through an interface.

FIG. 2 shows an API mediating inputs and outputs between a neuronal cell culture and multiple reference environments.

FIG. 3 shows an illustrative method for training a neuronal cell culture on multiple reinforcement learning tasks and comparing the performance.

FIG. 4 shows a computer architecture diagram of an electronic computing device capable of implementing aspects of the techniques and technologies presented herein.

### DETAILED DESCRIPTION

Neuronal cell cultures can be used as compute substrates as part of a computing system that includes both biological and electronic components. The use of neuronal cell cultures as compute substrates is discussed in U.S. patent application Ser. No. 18/367,392. One way that neuronal cell cultures can function as a compute substrate is by acting literally as a neural network-one implemented with actual neurons rather than mathematical relationships as in conventional machine learning. This use of neuronal cell cultures has been previously demonstrated in an experiment that trained approximately 800,000 brain cells grown in vitro to play a simple arcade game similar to Pong. See Brett J. Kagan et al., In vitro neurons learn and exhibit sentience when embodied in a simulated game-world, 23 Neuron 110, 3952-3969.e8 (2022).

Although this function and capability of neuronal cell cultures has been demonstrated, currently there is no established evaluation framework to evaluate biological learning. This limits knowledge about how learning occurs in neuronal cell cultures and the resources required for training. Most current techniques for studying neuronal cell cultures focus on structure rather than function. Readily measurable characteristics of limited value are used such as cell health and frequency or number of action potentials. Observing these types of superficial characteristics does not provide insight into the mechanisms of learning. The systems and techniques of this disclosure provide a way to systematically study biological learning implemented in a neuronal cell culture. The existence of standardized systems and techniques for interacting with neuronal cell cultures also makes it easier to use various neuronal cell cultures as testing platforms for the effects of drugs. Knowledge gained from this can improve understanding of how to design conventional (i.e., electronic) artificial intelligence as well as human and animal learning.

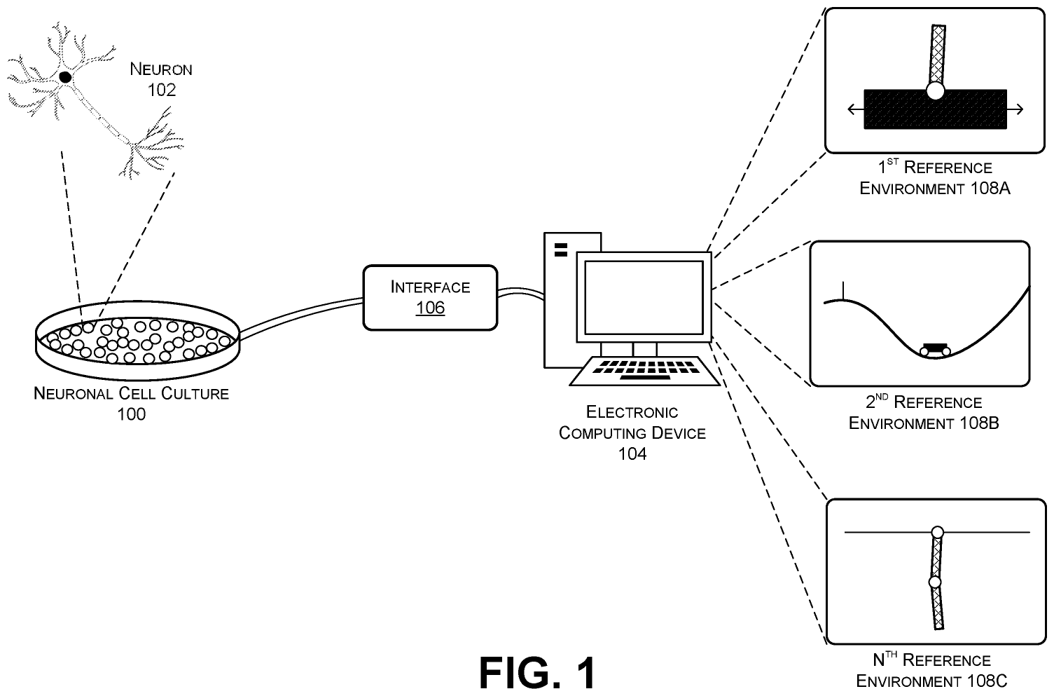

FIG. 1 shows a neuronal cell culture 100 that contains many thousands of neurons 102 connected to an electronic computing device 104 via an interface 106. Although only a single neuronal cell culture 100 is shown, multiple different neuronal cell cultures may be connected to the interface 106 in order to compare the abilities of different types of neuronal cell cultures. There are many techniques known to those of ordinary skill in the art for culturing neurons 102 to grow outside of a living organism. The neurons 102 may be grown from differentiated embryonic stem cells or induced pluripotent stem cells (iPSCs). The embryotic stem cells are guided using techniques known in the art (typically through chemical concentrations) to activate and/or inhibit genes that result in directed development into differentiated stem cells. Induced pluripotent stem cells (iPSCs) are a type of pluripotent stem cell that can be generated directly from a somatic cell. They are derived from skin or blood cells that have been reprogrammed back into an embryonic-like pluripotent state that enables the development of an unlimited source of any type of human cell needed. The neurons 102 may be human or from a non-human animal such as a primate or rodent. For example, the neurons 102 may be human induced pluripotent stem cells (hiPSCs). As a further example, the neurons 102 may be harvested from embryonic rodent brains. A neuronal cell culture 100 will typically contain a large number of individual neurons 102. In some implementations, the neurons 102 all come from the same source and are genetically identical. However, it is also possible to mix neurons 102 from different sources to create a neuronal cell culture 100 from two or more different types of neurons 102. Examples of suitable neurons 102 and techniques for creating neuronal cell cultures 100 are described in Cleber A. Trujillo et al., Complex Oscillatory Waves Emerging from Cortical Organoids Model Early Human Brain Network Development, 25 Stem Cell Reports 558 (2019).

The neuronal cell culture 100 may be a two-dimensional (2D) cell culture or a three-dimensional (3D) cell culture. In 2D cell cultures, cells are grown in a single layer on top of a flat surface, whereas in 3D cell cultures, the neurons 102 are grown in a 3D space. The formation of a cell monolayer in a 2D cell culture is faster and has lower reagent costs than in a 3D cell culture. However, 3D cell cultures mimic in vivo environments more closely and are typically longer lived than 2D cell cultures. A 3D cell culture is grown on a scaffolding that provides a structure for the cells to grow. The scaffolding may be created with sensors that become embedded in the cell culture as the subculture grows around the structures. An organoid is a specific type of 3D cell culture. Cancer tumorspheres are another example of a 3D cell culture.

An organoid is a miniaturized and simplified version of an organ produced in vitro in 3D that mimics the key functional, structural, and biological complexity of that organ. Organoids are derived from one or a few cells from a tissue, differentiated embryonic stem cells, or induced pluripotent stem cells, which can self-organize in three-dimensional culture owing to their intrinsic properties and/or extrinsic cues provided by the culture environment. Organoids are 3D cell cultures that contain organ-specific cell types that exhibit spatial organization and replicate some functions of the organ. Cortical organoids may exhibit cortical folds as well as vascularization.

Cortical organoids or brain organoids are one example of an organoid. Cortical organoids are derived from differentiated embryonic stem cells (ESCs) or iPSCs. Cortical organoids are created by culturing stem cells in a 3D rotational bioreactor and develop over months with cell types and cytoarchitectures that resemble an embryonic brain. The growth of a cortical organoid tends to reproduce the developmental path of the brain of a developing embryo. A cortical organoid grown from human cells is referred to as a human cortical organoid (HCO). Techniques for growing HCOs are known to those of ordinary skill in the art and described in Trujillo et al.

The neuronal cell culture 100 is communicatively connected to the electronic computing device 104 by an interface 106. The interface 106 is configured to provide a stimulus or input that is detected by and causes a response by at least some of the neurons 102 in the neuronal cell culture 100. Thus, the interface 106 may include one or more input devices that provide the input. There are many different ways to provide input to the neuronal cell culture 100 including, but not limited to, electricity, light, chemical, sound, motion, and heat. Electrical stimulation may be provided to the neurons 102 with an electrode such as, but not limited to, an electrode that is part of an MEA. Excitation caused by activation of an electrode may increase cell membrane potential to a point that causes the neuron 102 to discharge. Light may be provided by LEDs, a projector, a digital micromirror, or other light source. In some implementations, the light may be provided as strobing light, for example a light that strobes at a frequency of 10 Hz.

Chemical simulation may be provided by applying the chemical directly to neurons 102 in the neuronal cell culture 100. Chemical stimulation also be applied to vascularized portions of the neuronal cell culture 100 that can carry the chemical signal. The chemical may, for example, be applied in a solution, powder, or solid. The chemical may be applied by any conventional means for applying chemicals to cells in cell culture such as manual application with a dropper or pipette or automated application that uses laboratory robotics or microfluidics. Sound may be provided by one or more speakers or noisemakers. Motion may be provided by a vibrating tray, an agitator, or even by mechanically applying force (e.g., “poking”) neurons 104 in the neuronal cell culture 100. Heat may be applied by any conventional means for heating cells in cell culture such as the use of resistors to create localized warming, application of warm fluids, microwaves, and the like.

The interface 106 may include a component such circuitry that is configured to connect to and receive signals from multiple different types of stimulus modalities. Thus, a portion of the interface 106 may remain standard and is capable of being connected to multiple different ones out an MEA, a light source, a microfluidics device, agitator, a heater, or other types devices that can stimulate the neuronal cell culture 100. Standardization provided by the interface 106 may do things such as maintain the same spatial location of stimulus. For example, the interface 106 may be designed such that it will affect stimulus at the same location on the neuronal cell culture 100 regardless of which specific type of stimulus modality is used. Additionally, the interface 106 may also be configured to function to standardize or normalize behavior across different input modalities in other ways such as, but not limited to, maintaining the same relative strength of stimulus whether that stimulus is provided by light, electricity, or another modality.

The interface 106 may provide localized simulation to only a portion of the neurons 102 in the neuronal cell culture 100. Thus, input signals from the interface 106 can be provided to one or more specific regions of the neuronal cell culture 100. The regions of stimulation may be specified absolutely such as by specific coordinates designating a portion of the neuronal cell culture 100. The regions of stimulation may also be designated in reference to a location where output is detected such as an adjacent electrode or a set number of micrometers separated from the location of the output. It is also possible to stimulate the neuronal cell culture 100 on a subcellular level.

The interface 106 is configured to detect activity of at least some of the neurons 102 in the neuronal cell culture 100. Thus, the interface 106 may include one or more output devices that sense some signal or change of the neurons 102 such as action potentials. Depending on the specific type and configuration of the interface 106, output signals may be detected at one or more locations on the neuronal cell culture 100. There are many different ways to detect action potentials of a neuron 102.

In one implementation, the interface 106 includes one or more electrodes. The electrodes may be bidirectional electrodes capable of both stimulating the neurons and detecting activation of the neurons. For example, the interface 106 may be implemented as an MEA that detects electrical signals generated when there is an action potential in a neuron 102. In other implementations, thermal sensors may be used to detect changes in the temperature of the neurons 102 and optical sensors may be used to measure neural activity based on surface plasmon resonance. See Mitra Abedini et al., Recording Neural Activity Based on Surface Plasmon Resonance by Optical Fibers—A Computational Analysis, 12 Front. Comput. Neurosci., 16 October (2018). The input device and output device that make up the interface 106 may be different devices and use different techniques for interfacing with the neuronal cell culture 100. For example, the input device may apply a chemical to the neuronal cell culture 100 to provide input signals while the output device detects temperature changes.

In some implementations, the interface 106 contains a single device that functions as both the input device and the output device. For example, an MEA may serve as both an input device and an output device. A single bidirectional electrode may also serve as both an input device and an output device. When neurons 102 in the neuronal cell culture 100 generate an action potential, they produce electrical signals that can be detected by electrodes in the MEA. Examples of suitable MEAs, and techniques for growing a neuronal cell culture 100 on an MEA are known to those of ordinary skill in the art and described in Trujillo et al., Francesca Puppo et al., Super-Selective Reconstruction of Causal and Direct Connectivity With Application to in vitro iPSC Neuronal Networks, 15 Frontiers in Neuroscience (2021); and Kagan et al.

In some implementations, the interface 106 includes circuitry that is used to couple the input device and output device with the electronic computing device 104. The specific type and function of the circuitry will depend upon the type of input device and output device used in the interface 106. If the interface 106 is implemented as an MEA, the circuitry provides an electrical and communicative connection between the electrodes of the MEA and the electronic computing device 104. The circuitry may be any type of circuitry conventionally used to control an MEA or an alternative type of input device and output device. Control systems for MEAs are known to those of ordinary skill in the art and described in Trujillo et al., Puppo et al., Kagan et al., and Tianyi Chen et al., Discovering a Change Point and Piecewise Linear Structure in a Time Series of Organoid Networks via the Iso-Mirror, Applied Network Science 8:45 (2023). Control systems, software, MEAs, and accompanying circuitry are available from commercial sources including Axion BioSystems (Atlanta GA, USA).

The electronic computing device 104 can be any type of conventional computing device such as a desktop computer, laptop computer, tablet computer, smartphone, or the like. The electronic computing device 104 may also be physically located at a distance from the neuronal cell culture 100. For example, the electronic computing device 104 may be a network-accessible or cloud-based computing device with physical components spread across multiple different locations. In an implementation, the interface 106 is connected to a network and signals from the interface 106 are conveyed through the network to the electronic computing device 104.

The electronic computing device 104 includes software components that drive the interface 106 as well as interpret signals received from the interface 106. The software converts input data into instructions that cause the interface 106 to generate a specific input signal to the neuronal cell culture 100. Output signals detected by the interface 106 and provided to the electronic computing device 104 are interpreted by the software and converted into output data. Thus, the software is used to encode information in a way that can be provided to the neuronal cell culture 100 and to decode patterns of action potentials detected by the interface 106. Many of these functions performed by software can also be implemented by firmware or specialized hardware.

The electronic computing device 104 may maintain multiple reference environments 108(1-N). The reference environments 108(1-N) may be simulated environments that provide goal-oriented tasks similar to those used in classic control-based reinforcement learning tasks, such as Cart Pole, Pendulum, or Arcobot. The neuronal cell culture 100 is trained, together with accompanying components on the electronic computing device 104, to perform the reinforcement learning tasks provided by each of the reference environments 108(1-N). The reference environments 108(1-N) may be any type of environment that reinforcement learning algorithms can be trained to solve. However, in some implementations the reference environments 108(1-N) provide classic control tasks that are inherently linked to innate motor and movement control, with a small and manageable set of states.

The interface 106, electronic computing device 104, and the reference environments 108(1-N) provide a standardized framework to conduct multiple different learning experiments using a supervision reward system to test theories of learning mechanisms. The availability of multiple pre-built environments allows researchers and developers to quickly prototype and test neuronal cell cultures 100 in a variety of environments, without the need to build a custom simulation framework for each problem. This makes it possible to obtain a more diverse view of how a specific neuronal cell culture 100 performs as a compute substrate by testing it on more than one reinforcement learning task. This standardized framework can be used to benchmark methods and implementations (both on the electronic computing device 104 and with neuronal cell cultures 100) and compare results. Comparison of results can advance the use of neuronal cell cultures 100 as compute substrates by identifying such things as the types of cell, cell cultures, and training techniques that provide the best performance.

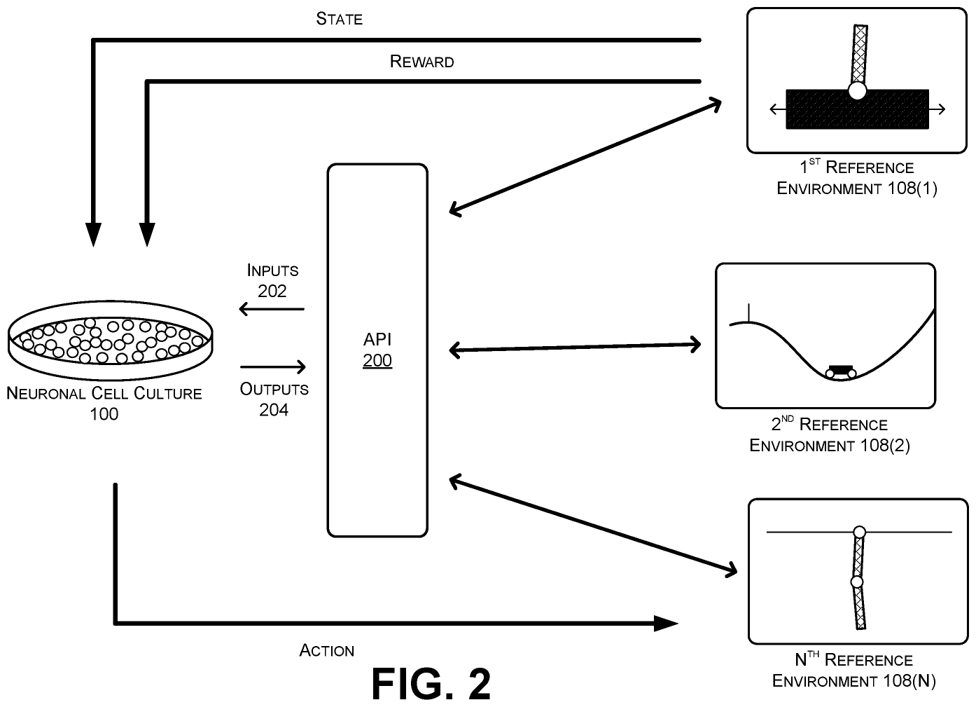

FIG. 2 shows an Application Programming Interface (API) 200 mediating the flow of information between a neuronal cell culture 100 and multiple reference environments 108(1-N). The API 200 is different from a conventional API because it does not facilitate communication between two software programs but abstracts the communication between the neuronal cell culture 100 and reference environments 108(1-N). Both the inputs 202 provided to the neuronal cell culture 100 and the outputs 204 detected from the neuronal cell culture 100 can be handled in a standardized way by the API 200. This makes it possible for the same neuronal cell culture 100 to be tested or trained on different ones of the reference environments 108(1-N) with minimal additional effort. Alternatively, with this framework it is also possible for different neuronal cell cultures to be connected via the API 200 with the same reference environment 108 in a standardized and reproducible way.

The inputs 202 may include things such as the type, timing, and location of stimulus provided to the neuronal cell culture 100. When connected to an MEA, the API 200 may specify a standard technique for providing signals from input devices to and detecting signals from sensors. When a MEA functions as the input device and sensor, the API 200 will mediate signals going to and from the MEA. For example, the voltage of electrical stimulation provided by an electrode may be standardized through the API 200. Additionally, the API 200 may standardize the frequency per unit time of detecting “spikes” or sudden temporary changes in voltage. As a further example, a correspondence between a location of a stimulus on the neuronal cell culture 100 and a state of a reference environment 108 can be standardized. This could be implemented, for example, on an array of 20×20 electrodes sampling voltage at 1000 Hz in which a relationship between the voltages, frequency detected, and individual electrodes are correlated with a reference environment 108. The outputs 204 include changes in the neuronal cell culture 100 such as activation potentials detected by sensors. The API 200 may standardize where outputs are detected and interpretations of output patterns.

The neuronal cell culture 100 can participate in reinforcement learning by interacting with the API 200. The reinforcement learning process involves an agent taking actions in an environment and receiving feedback in the form of a reward signal. The agent is implemented at least in part by the neuronal cell culture 100. Conventional software or AI components on an electronic computing device may function together with the neuronal cell culture 100 to create the agent. The agent uses this feedback to improve its decision-making process, learning over time which actions lead to the highest rewards. Reward in this context can refer to positive feedback as well as negative feedback. The goal of the agent is to find an optimal policy, which specifies the best action to take in each state to maximize the expected cumulative reward. Information about the current state of a reference environment 108 is provided to the neuronal cell culture 100. Thus, inputs 202 may provide both a reward signal and state information.

The reinforcement learning process can be thought of as a trial-and-error search, where the agent explores different actions and observes their consequences in a reference environment 108 to learn which actions lead to the highest rewards. Successful activity of an agent balances exploration (trying new actions) with exploitation (using actions that are known to yield high rewards). Reinforcement learning is based on the hypothesis that all goals can be described by the maximization of expected cumulative reward. The agent learns to sense and alter the state of the environment using its actions to derive maximal reward.

In this context, the environment where the agent acts is the reference environment 108 which may be a virtual world provided by an electronic computing device. The reference environment 108 is the world in which the agent operates. The state is the current situation of the agent and the environment. State provides information about the current situation that the agent can use to decide what action to take next. The state can include any information that is relevant to the agent's decision-making process, such as the current position of objects in the environment or the current score in a game. The state changes over time as the agent takes actions and interacts with the environment.

The reward is feedback from the environment. One way to provide a reward is to use Fristo's Free Energy Principle as a learning mechanism through supervised training. This technique was used by Kagan et al. to train brain cells grown in vitro to play a game similar to Pong. In neuroscience, the Free Energy Principle is based on the Bayesian idea of the brain as an “inference engine.” Under this principle, systems pursue paths of least surprise, or equivalently, minimize the difference between predictions based on their model of the world and their sense and associated perception. This difference is quantified by variational free energy and is minimized by continuous correction of the world model of the system, or by making the world more like the predictions of the system. Providing stimulation to the neurons that was “surprising” acted as a negative reward and providing structured stimulation that was “familiar” acted as a positive reward.

A reward can be provided by any signal that can be sensed by the neurons of a neuronal cell culture 100. The reward can be implemented with a deterministic signal. A deterministic signal is a type of signal in which the value of the signal at any given time can be predicted exactly. This means that the signal follows a well-defined mathematical function or rule, and its future values can be calculated based on its past values. The type of signal is one factor that can be varied in reinforcement learning. The reward could be provided by an electric signal generated at an electrode or a chemical signal created by applying a chemical to the cell culture. For example, the application of a chemical such as glucose that may be beneficial to the neurons could be used as a reward. Examples of techniques for applying chemicals to neuronal cell cultures 100 and observing the response are described in U.S. patent application Ser. No. 18/236,366. The chemicals may be drugs applying them to a neuronal cell culture 100 may be used to study the effects of the drug on neurons. By having the neuronal cell culture 100 connected through the API 200 to a reference environment 108 it is possible to observe how presence of the drug affects the neuronal cell culture's ability to learn or perform a reinforcement learning task. Use of the API 200 allows the effects of numerous different drugs on multiple different types of neuronal cell cultures 100 to be compared in a standardized and reproducible way.

Depending upon the action that the neuronal cell culture 100 takes in the environment, it may or may not receive the reward. The reward used for reinforcement learning encourages the desired behavior from the neuronal cell culture 100. Thus, reinforcement learning can structurally teach a set of neurons how to interact and achieve behavior that is objectively deemed “good” according to the context of a reinforcement learning task in an environment. With the API 200, the type of signal used to signal a reward in the location on the neuronal cell culture 100 where the signal is applied to be standardized. The reinforcement learning gym provides a framework that can be used comparing different combinations of inputs 202 and rewards.

In reinforcement learning, policy is a method to map the agent's state to actions. Policy may be learned by the neuronal cell culture 100 by interacting with a reference environment 108 and receiving a reward. Value is the expected reward that the agent would receive by taking an action in a particular state. Updating the reward function informs the system if the agent has made a good move or a bad move in the context of the reference environment 108. The way of providing inputs 202 about the state of the reference environment 108 may also be standardized.

In a reinforcement learning gym, the reference environments 108 may be simulated environments that are designed to simulate a wide range of scenarios, which can be based on real-world environments or virtual environments, and problems that reinforcement learning algorithms can be trained to solve. A reinforcement learning gym allows researchers and developers to quickly prototype and test a neuronal cell culture 100 in a variety of environments, without the need to build a custom simulation framework for each problem. The API 200 provides a standardized way for interacting with the reference environments 108, which makes it easy to switch between different reference environments 108. The standardization also makes it easier to compare the performance of different types of neuronal cell cultures 100.

Testing reinforcement learning on neuronal cell cultures 100 can be used to identify the best cell type to use for a particular application (e.g., human or mouse neurons). It could also be used to evaluate how long an organoid should be cultured before it is trained such as three or six months. Additionally, training a neuronal cell culture 100 to perform reinforcement learning tasks can be used to determine how to best encode information, represent state variables, and to deliver rewards. Additionally, a framework for comparing different reinforcement learning tasks could be used to identify the best network structure for a particular application.

Reactions of the neuronal cell culture 100 are the outputs 204 passed through the API 200 to understand whether the neuronal cell culture 100 is successfully performing a reinforcement learning task. Thus, the outputs 204 are configured to indicate action taken by the neuronal cell culture 100 as an agent. The API 200 may interpret the outputs 204 by using region of interest mapping. Region of interest mapping assigns value or particular meaning to activity in neuronal cell culture based on the location of that activity. It is possible to detect which regions of the neuronal cell culture 100 are changed and correlate the region with an environmental state update. For example, if a first region of the neuronal cell culture 100 increases in activity an object in the reference environment 108 is moved to the left while an increase in activity in a different region moves the object to the right. The results of changes in the reference environment 108, the current state of the environment, is sent back to the neuronal cell culture 100 as input 202. Depending on the action taken by the neuronal cell culture 100 as an agent, a reward may be provided. This cycle is repeated multiple times during training as the neuronal cell culture 100 interacts with the environment. With this feedback cycle, the neuronal cell culture 100 as agent learns how to perform a reinforcement learning task in the reference environments 108.

By providing rewards and informing the neuronal cell culture 100 about the state of the reference environment, new neural pathways can be formed and existing pathways strengthened so that the neuronal cell culture 100 begins to behave differently. This is how the neuronal cell culture 100 learns. Thus, a neuronal cell culture can be trained in a manner analogous to a neural network implemented in a conventional electronic computer. The effect of training can be evaluated behaviorally by identifying if the neuronal cell culture 100 gains skills and becomes increasingly able to perform the reinforcement learning task.

### Methods

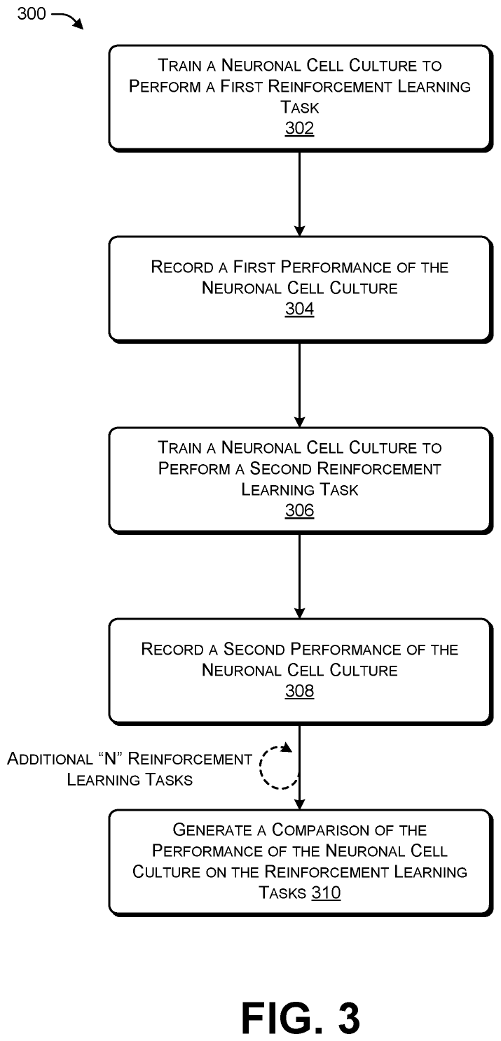

FIG. 3 shows a method 300 of training a neuronal cell culture on multiple reinforcement learning tasks and comparing the performance on each task. Method 300 may be performed by any of the systems and components shown in FIGS. 1 and 2.

At operation 302, a neuronal cell culture is trained to perform a first reinforcement learning task. The neuronal cell culture may be any type of neuronal cell culture described in this disclosure. For example, the neuronal cell culture may be a two-dimensional (2D) cell culture or a three-dimensional (3D) cell culture such as an organoid. The neuronal cell culture may be formed from differentiated embryonic stem cells or induced pluripotent stem cells. The neurons in the neuronal cell culture may come from a human, a mouse, or another animal.

The first reinforcement learning task may be any type of task that can be learned by a machine learning algorithm applying reinforcement learning. For example, the task may be a classic control task used in reinforcement learning such as Cart Pole, Mountain Car, Acrobot, Pendulum, or the like. More generally, the tasks may be any goal-oriented behavioral tasks. Goal-oriented behavioral tasks include classic control tasks such as those mentioned above as well as other types of task in which the agent interacts with an environment to achieve a predefined objective state. In some implementations, a reference environment that provides the first reinforcement learning task is a simulated environment maintained on an electronic computing device. However, the reinforcement learning tasks are not limited to goal-oriented behavioral tests. For example, other types of reinforcement learning tasks include maintenance tasks in which the neuronal cell culture maintains a current state as long as possible. The state could be something such as a condition of the neurons (e.g., staying alive).

The neuronal cell culture is trained on the first reinforcement learning task by using a standard protocol such as an API to communicatively connect the neuronal cell culture to an electronic computing device. An interface connected to the electronic computing device provides inputs to the neuronal cell culture and receives outputs from the neuronal cell culture. The protocol is standardized in that the specific hardware and techniques for providing inputs and detecting outputs, as well as the information contained in those inputs and outputs, are the same or similar across multiple reinforcement learning tasks.

At operation 304, a first performance of the neuronal cell culture on the first reinforcement learning task is recorded. The performance of the neuronal cell culture may be recorded in the conventional memory of an electronic computing device. Performance on the task can be evaluated in any of the same ways used for conventional, purely electronic-based reinforcement learning. For example, the performance may be the success or failure at completing the task. This could be recorded as a binary value. Performance may also be a level of competence at the task—how well the neuronal cell culture (and accompanying electronic computing systems) can perform the task. Competence may be recorded by gradations such as low, medium, high, or a numerical value such as a value between 0 and 10. Competency may also evaluate not just how well the neuronal cell culture performs when trained, but also how well the neuronal cell culture learns. Thus, the speed of learning the task may be another type of performance that is recorded. The speed of learning may be measured in time or training cycles/iterations.

At operation 306, the neuronal cell culture is trained to perform a second reinforcement learning task. The second reinforcement learning task is different than the first reinforcement learning task. Because the same standard protocol is used for the second reinforcement learning task as the first reinforcement task, a user is able to easily test the neuronal cell culture on the first reinforcement learning task followed by switching to testing on the second reinforcement learning task. Certain details of the inputs may be different because the neuronal cell culture is learning a different task, but the hardware and techniques for communication via the interface to the electronic computing device are the same. The standardization also makes it possible to easily test different neuronal cell cultures on the same reinforcement learning task.

Part of the standard protocol for training the neuronal cell culture to perform a reinforcement learning task may be the reward given during training. As mentioned above, there are many ways to provide an input to a neuronal cell culture that is interpreted as a reward. For example, the reward may be electrical stimulation, light stimulation, or chemical stimulation applied to the neuronal cell culture. Other types of stimulation may also function as a reward for training. Because of the standardization, in some implementations, the reward used to train the neuronal cell culture to perform the first reinforcement learning task is the same as the reward used to train the neuronal cell culture to perform the second reinforcement learning task.

At operation 308, a second performance of the neuronal cell culture on the second reinforcement learning task is recorded. Performance on the second reinforcement learning task may be measured in any of the same ways as for the first reinforcement learning task. Preferably, the same metrics are recorded to enable direct comparisons. The second performance on the second reinforcement learning task may be recorded in the same file or datastore as the first performance on the first reinforcement learning task.

The neuronal cell culture may be trained to perform “N” additional (i.e., more than 1) reinforcement learning tasks where N may be 1 (performing two tasks) or a larger integer. If N is 2, then the neuronal cell culture is trained to perform a third (i.e., 1+2) reinforcement learning task using the same standard protocols for providing inputs and receiving outputs via the interface connected to the electronic computing device. The third reinforcement learning task is different from the first reinforcement learning task and the second reinforcement learning task. A third performance of the neuronal cell culture on the third reinforcement learning task will then be recorded in the same way as for the first and second reinforcement learning tasks. This may be repeated for any number of different reinforcement learning tasks.

As part of a reinforcement learning “gym,” there may be many pre-built environments and reinforcement learning tasks available to use for evaluating a neuronal cell culture. Testing the learning and performance of the neuronal cell culture on multiple different tasks provides a more detailed and robust basis for evaluating the suitability of the neuronal cell culture as a compute substrate than testing on just a single task. A given neuronal cell culture may be good at performing certain types of reinforcement learning tasks but have difficulty with other types of tasks. This would not be apparent if the neuronal cell culture was tested on only one task (e.g., playing a game like Pong).

At operation 310, a comparison of the performance of the neuronal cell culture on the reinforcement learning tasks is generated. The comparison may include the first performance of the neuronal cell culture on the first reinforcement learning task, the second performance of the neuronal cell culture on the second reinforcement learning task, and the performance on any additional reinforcement learning tasks. This comparison may directly compare the same performance metric across multiple tasks (e.g., success or failure at each task). It may also include mathematical and statistical evaluations based on the performance (e.g., average learning time). Once generated, the comparison may be stored (in a conventional electronic computer system) and/or displayed on an output device such as a monitor. This comparison provides a record and a basis for a user to evaluate the performance of the neuronal cell culture on not just one but multiple different reinforcement learning tasks.

Multiple different neuronal cell cultures can be run through the same set of reinforcement learning tasks in the “gym.” This provides a standard way to identify baseline performance for neuronal cell cultures. Comparing the baseline of different neuronal cell cultures may provide insights into which types of neuronal cell cultures and methods of training are most effective in creating a biologically-based compute substrate. Things that can be varied between different neuronal cell cultures include the types of cells, the number of cells, the architecture of the cell culture (e.g., 2D or 3D), the culture media, and the length of development before training. The mechanics of training and connecting a neuronal cell culture to an electronic computing device can also be varied. For example, things such as the location of sensors in the neuronal cell cultures and the type of device used to stimulate and provide inputs to the neuronal cell cultures can be varied.

### Computing Devices and Systems

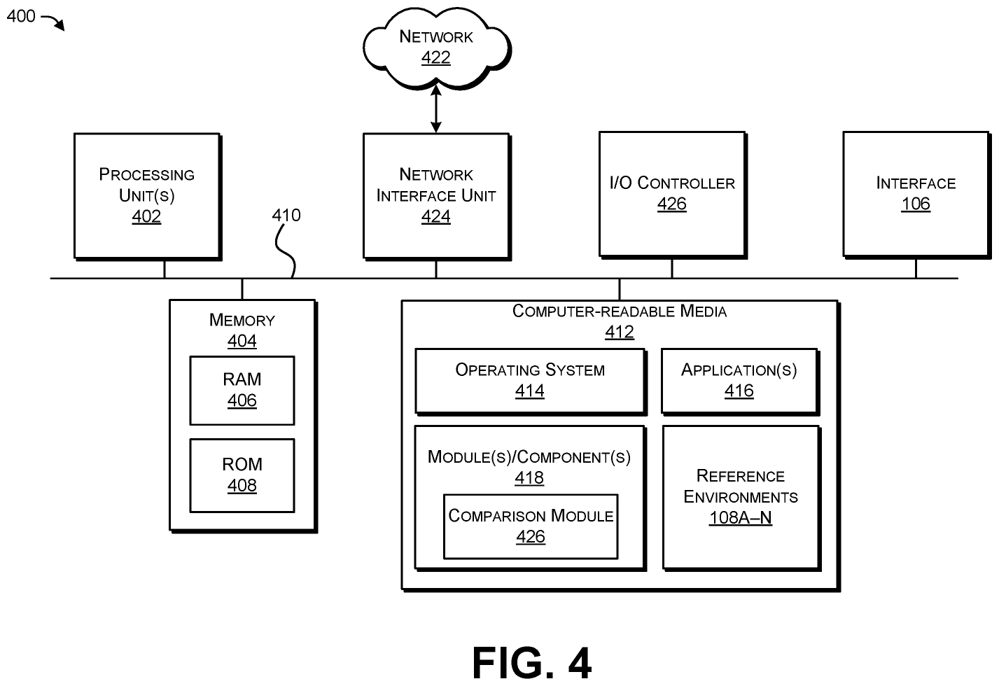

FIG. 4 shows details of an example computer architecture 400 for an electronic computing device such as the electronic computing device 104 introduced in FIG. 1. The computer architecture 400 illustrated in FIG. 4 includes processing unit(s) 402, a memory 404, including a random-access memory 406 (“RAM”) and a read-only memory (“ROM”) 408, and a system bus 410 that couples the memory 404 to the processing unit(s) 402. The processing unit(s) 402 include one or more hardware processors and may also comprise or be part of a processing system. In various examples, the processing unit(s) 402 of the processing system are distributed. Stated another way, one processing unit 402 may be located in a first location (e.g., a rack within a datacenter) while another processing unit 402 of the processing system is located in a second location separate from the first location.

The processing unit(s) 402 can represent, for example, a CPU-type processing unit, a GPU-type processing unit, a field-programmable gate array (FPGA), another class of digital signal processor (DSP), or other hardware logic components that may, in some instances, be driven by a CPU. For example, illustrative types of hardware logic components that can be used include Application-Specific Integrated Circuits (ASICs), Application-Specific Standard Products (ASSPs), System-on-a-Chip Systems (SOCs), Complex Programmable Logic Devices (CPLDs), and the like.

A basic input/output system containing the basic routines that help to transfer information between elements within the computer architecture 400, such as during startup, is stored in the ROM 408. The computer architecture 400 further includes a computer-readable media 412 for storing an operating system 414, application(s) 416, modules/components 418, and other data described herein. The application(s) 416 and the module(s)/component(s) 418 may implement communication of signals between the electronic computing device and a neuronal cell culture. The other data may include the reference environments 108A-N used for testing the neuronal cell cultures.

The modules/components 418 may include a comparison module 420. The comparison module 420 tracks the results of a neuronal cell culture on multiple reinforcement learning tasks. The comparison module 420 may also track the results of multiple different neuronal cell cultures across the multiple reinforcement learning tasks. The comparison module 420 may identify common elements in the performance of one or more neuronal cell cultures and provides comparisons of the common elements (e.g., number of cycles required to solve the mountain car task). The comparison module 420 may also calculate differences and statistics based on the comparisons. For example, the comparison module 420 may determine if a difference is statistically significant. The comparison module 420 may be configured to present a comparison of results obtained by the neuronal cell culture on the multiple reinforcement learning tasks such as by causing the computer architecture 400 to display the comparison on a user interface.

The computer-readable media 412 is connected to the processing unit(s) 402 through a storage controller connected to the bus 410. The computer-readable media 412 provides non-volatile storage for the computer architecture 400. The computer-readable media 412 may be implemented as a mass storage device, yet it should be appreciated by those skilled in the art that computer-readable media can be any available computer-readable storage medium or communications medium that can be accessed by the computer architecture 400.

Computer-readable media includes computer-readable storage media and/or communication media. Computer-readable storage media can include one or more of volatile memory, nonvolatile memory, and/or other persistent and/or auxiliary computer storage media, removable and non-removable computer storage media implemented in any method or technology for storage of information such as computer-readable instructions, data structures, program modules, or other data. Thus, computer storage media includes tangible and/or physical forms of media included in a device and/or hardware component that is part of a device or external to a device, including RAM, static random-access memory (SRAM), dynamic random-access memory (DRAM), phase-change memory (PCM), ROM, erasable programmable read-only memory (EPROM), electrically erasable programmable read-only memory (EEPROM), flash memory, compact disc read-only memory (CD-ROM), digital versatile disks (DVDs), optical cards or other optical storage media, magnetic cassettes, magnetic tape, magnetic disk storage, magnetic cards or other magnetic storage devices or media, solid-state memory devices, storage arrays, network-attached storage, storage area networks, hosted computer storage or any other storage memory, storage device, and/or storage medium that can be used to store and maintain information for access by a computing device.

In contrast to computer-readable storage media, communication media can embody computer-readable instructions, data structures, program modules, or other data in a modulated data signal, such as a carrier wave, or other transmission mechanism. As defined herein, computer-readable storage medium does not include communication medium. That is, computer-readable storage media does not include communications media and thus excludes media consisting solely of a modulated data signal, a carrier wave, or a propagated signal, per se.

According to various configurations, the computer architecture 400 may operate in a networked environment using logical connections to remote computers through the network 422. The computer architecture 400 may connect to the network 422 through a network interface unit 424 connected to the bus 410. In some implementations, the interface 106 may be connected through the network 422 to the computer architecture 400. An I/O controller 426 may also be connected to the bus 410 to control communication in input and output devices. The interface 106 may, in some implementations, be connected through the I/O controller 426. Alternatively, the interface 106 may be connected to the bus 410 without going through the I/O controller 426. Thus, the computer architecture 400 may be communicatively coupled to the neuronal cell culture through the interface 106.

The interface 106, when configured for interfacing with an MEA, may include a bandpass filter and adaptive threshold spike detector as well as potentially a spike sorter. For example, the bandpass filter may be set to 10-25,000 Hz. In another implementation, the bandpass filter may be set to 0.1 Hz to 5 kHz. The adaptive threshold spike detector may be set to 5.5× standard deviations. In one implementation, the circuitry may include a 2^{nd} order high-pass Bessel filter with 100 Hz cut-off followed by a 1^{st} order low-pass Bessel filter with 1 Hz cut-off. Raw data after being passed through the interface 106 can be acquired and processed with software such as the Maestro recording system and Axion Integrated Studio available from Axion Biosystems. Alternative software that may be used includes the AxIS Software Spontaneous Neural Configuration also from Axion Biosystems.

It should be appreciated that the software components described herein may, when loaded into the processing unit(s) 402 and executed, transform the processing unit(s) 402 and the overall computer architecture 400 from a general-purpose computing system into a special-purpose computing system customized to facilitate the functionality presented herein. The processing unit(s) 402 may be constructed from any number of transistors or other discrete circuit elements, which may individually or collectively assume any number of states. More specifically, the processing unit(s) 402 may operate as a finite-state machine, in response to executable instructions contained within the software modules disclosed herein. These computer-executable instructions may transform the processing unit(s) 402 by specifying how the processing unit(s) 402 transitions between states, thereby transforming the transistors or other discrete hardware elements constituting the processing unit(s) 402.

### Illustrative Embodiments

The following clauses describe multiple possible embodiments for implementing the features described in this disclosure. The various embodiments described herein are not limiting nor is every feature from any given embodiment required to be present in another embodiment. Any two or more of the embodiments may be combined together unless context clearly indicates otherwise. As used herein in this document “or” means and/or. For example, “A or B” means A without B, B without A, or A and B. As used herein, “comprising” means including all listed features and potentially including addition of other features that are not listed. “Consisting essentially of” means including the listed features and those additional features that do not materially affect the basic and novel characteristics of the listed features. “Consisting of” means only the listed features to the exclusion of any feature not listed.

Clause 1. A system that may be implemented in software only comprising:
a plurality of reference environments (108) each configured to provide a reinforcement learning task; and
an Application Programming Interface (API) (200) configured to provide inputs (202) to and receive outputs (204) from a neuronal cell culture (100), wherein the inputs are configured to provide the neuronal cell culture information about the reference environments and a reward, and the outputs are configured to indicate an action taken by the neuronal cell culture as an agent.

Clause 2. The system of clause 1, wherein the reference environments are simulated environments on an electronic computing device.

Clause 3. The system of clause 1 or 2, wherein the reinforcement learning task is a goal-oriented behavioral task.

Clause 4. The system of any one of clauses 1 to 3, wherein the API specifies a format of the inputs and a format of the outputs that is used by all of the plurality of reference environments.

Clause 5. The system of clause 4, wherein the API specifies a standard technique for providing signals to and detecting signals from input devices and sensors.

Clause 6. The system of clause 4, wherein the API specifies locations and voltages of electrical signals provided to and detected from the neuronal cell culture by a multielectrode array (MEA).

Clause 7. The system of any one of clauses 1 to 5, wherein the reward is electrical stimulation, light stimulation, or chemical stimulation applied to the neuronal cell culture.

Clause 8. The system of any one of clauses 1 to 7, further comprising the neuronal cell culture and an interface configured to convey the inputs and outputs between an electronic computing device and the neuronal cell culture.

Clause 9. A system comprising:
a neuronal cell culture (100):
an interface (106) configured to communicatively couple the neuronal cell culture to an electronic computing device (104);
the electronic computing device comprising:
   a processing unit (402);
   a memory (404);
   a first reference environment (108(1)), implemented by the processing unit, that is configured to provide a first reinforcement learning task to the neuronal cell culture; and
   a second reference environment (108(2)), implemented by the processing unit, that is configured to provide a second reinforcement learning task to the neuronal cell culture.

Clause 10. The system of clause 9, wherein the neuronal cell culture is a two-dimensional (2D) cell culture or a three-dimensional (3D) cell culture comprising differentiated embryonic stem cells or induced pluripotent stem cells.

Clause 11. The system of clause 9 or 10, wherein the interface comprises an input device configured to stimulate neurons in the neuronal cell culture and an output device configured to detect activation potentials of neurons in the neuronal cell culture.

Clause 12. The system of any one of clauses 9 to 11, wherein the electronic computing device further comprises an API that specifies a standard technique for providing inputs to and receiving outputs from the neuronal cell culture via the interface, wherein the API is the same for the first reference environment and the second reference environment.

Clause 13. The system of any one of clauses 9 to 12, wherein the electronic computing device further comprises a comparison module configured to present a comparison of results obtained by the neuronal cell culture on the first reinforcement learning task and on the second reinforcement learning task.

Clause 14. The system of any one of clauses 9 to 14, wherein the electronic computing device further comprises a third reference environment, implemented by the processing unit, that is configured to provide a third reinforcement learning task to the neuronal cell culture.

Clause 15. A method related to training on multiple tasks comprising:
training (302) a neuronal cell culture (100) to perform a first reinforcement learning task by communicating with the neuronal cell culture using a standard protocol for providing inputs (202) and receiving outputs (204) via an interface (106) connected to an electronic computing device (104);
recording (304) a first performance of the neuronal cell culture on the first reinforcement learning task;
training (306) the neuronal cell culture to perform a second reinforcement learning task by communicating with the neuronal cell culture using the standard protocol for providing inputs and receiving outputs via the interface connected to the electronic computing device; and
recording (308) a second performance of the neuronal cell culture on the second reinforcement learning task.

Clause 16. The method of clause 15, wherein the neuronal cell culture is a two-dimensional (2D) cell culture or a three-dimensional (3D) cell culture comprising differentiated embryonic stem cells or induced pluripotent stem cells.

Clause 17. The method of clause 15 or 16, wherein the first performance or the second performance comprises success or failure at completing a reinforcement learning task, a level of competence at the reinforcement learning task, or a speed of learning the reinforcement learning task.

Clause 18. The method of any one of clauses 15 to 17, wherein a reward used to train the neuronal cell culture to perform the first reinforcement learning task is the same as the reward used to train the neuronal cell culture to perform the second reinforcement learning task.

Clause 19. The method of clause 18, wherein the reward is electrical stimulation, light stimulation, or chemical stimulation applied to the neuronal cell culture.

Clause 20. The method of any one of clauses 15 to 19, further comprising generating a comparison of the first performance of the neuronal cell culture on the first reinforcement learning task to the second performance of the neuronal cell culture on the second reinforcement learning task.

Clause 21. The method of claim 14, further comprising:
training the neuronal cell culture to perform a third reinforcement learning task by communicating with the neuronal cell culture using the standard protocol for providing inputs and receiving outputs via the interface connected to the electronic computing device; and
recording a third performance of the neuronal cell culture on the third reinforcement learning task.

### CONCLUSION

While certain example embodiments have been described, including the best mode known to the inventors for carrying out the invention, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein. Skilled artisans will know how to employ such variations as appropriate, and the embodiments disclosed herein may be practiced otherwise than specifically described. The accompanying claims and their equivalents are intended to cover such forms or modifications as would fall within the scope and spirit of certain of the inventions disclosed herein.

The terms “a,” “an,” “the” and similar referents used in the context of describing the invention are to be construed to cover both the singular and the plural unless otherwise indicated herein or clearly contradicted by context. The terms “based on,” “based upon,” and similar referents are to be construed as meaning “based at least in part” which includes being “based in part” and “based in whole,” unless otherwise indicated or clearly contradicted by context. The terms “portion,” “part,” or similar referents are to be construed as meaning at least a portion or part of the whole including up to the entire noun referenced.

It should be appreciated that any reference to “first,” “second,” etc. elements within the Summary and/or Detailed Description is not intended to and should not be construed to necessarily correspond to any reference of “first,” “second,” etc. elements of the claims. Rather, any use of “first” and “second” within the Summary, Detailed Description, and/or claims may be used to distinguish between two different instances of the same element (e.g., two different sensors).

In closing, although the various configurations have been described in language specific to structural features and/or methodological acts, it is to be understood that the subject matter defined in the appended representations is not necessarily limited to the specific features or acts described. Rather, the specific features and acts are disclosed as example forms of implementing the claimed subject matter.

Furthermore, references have been made to publications, patents and/or patent applications throughout this specification. Each of the cited references is individually incorporated herein by reference for its particular cited teachings as well as for all that it discloses.

1. A system comprising:
a plurality of reference environments each configured to provide a reinforcement learning task; and
an Application Programming Interface (API) configured to provide inputs to and receive outputs from a neuronal cell culture, wherein the inputs are configured to provide the neuronal cell culture information about the reference environments and a reward, and the outputs are configured to indicate an action taken by the neuronal cell culture as an agent.

2. The system of claim 1, wherein the reference environments are simulated environments on an electronic computing device.

3. The system of claim 1, wherein the reinforcement learning task is a goal-oriented behavioral task.

4. The system of claim 1, wherein the API specifies a format of the inputs and a format of the outputs that is used by all of the plurality of reference environments.

5. The system of claim 4, wherein the API specifies a standard technique for providing signals to and detecting signals from input devices and sensors.

6. The system of claim 1, wherein the reward is electrical stimulation, light stimulation, or chemical stimulation applied to the neuronal cell culture.

7. The system of claim 1, further comprising the neuronal cell culture and an interface configured to convey the inputs and outputs between an electronic computing device and the neuronal cell culture.

8. A system comprising:
a neuronal cell culture:
an interface configured to communicatively couple the neuronal cell culture to an electronic computing device;
the electronic computing device comprising:
a processing unit;
a memory;
a first reference environment, implemented by the processing unit, that is configured to provide a first reinforcement learning task to the neuronal cell culture; and
a second reference environment, implemented by the processing unit, that is configured to provide a second reinforcement learning task to the neuronal cell culture.

9. The system of claim 8, wherein the neuronal cell culture is a two-dimensional (2D) cell culture or a three-dimensional (3D) cell culture comprising differentiated embryonic stem cells or induced pluripotent stem cells.

10. The system of claim 8, wherein the interface comprises an input device configured to stimulate neurons in the neuronal cell culture and an output device configured to detect activation potentials of neurons in the neuronal cell culture.

11. The system of claim 8, wherein the electronic computing device further comprises an API that specifies a standard technique for providing inputs to and receiving outputs from the neuronal cell culture via the interface, wherein the API is the same for the first reference environment and the second reference environment.

12. The system of claim 8, wherein the electronic computing device further comprises a comparison module configured to present a comparison of results obtained by the neuronal cell culture on the first reinforcement learning task and on the second reinforcement learning task.

13. The system of claim 8, wherein the electronic computing device further comprises a third reference environment, implemented by the processing unit, that is configured to provide a third reinforcement learning task to the neuronal cell culture.

14. A method comprising:
training a neuronal cell culture to perform a first reinforcement learning task by communicating with the neuronal cell culture using a standard protocol for providing inputs and receiving outputs via an interface connected to an electronic computing device;
recording a first performance of the neuronal cell culture on the first reinforcement learning task;
training the neuronal cell culture to perform a second reinforcement learning task by communicating with the neuronal cell culture using the standard protocol for providing inputs and receiving outputs via the interface connected to the electronic computing device; and
recording a second performance of the neuronal cell culture on the second reinforcement learning task.

15. The method of claim 14, wherein the neuronal cell culture is a two-dimensional (2D) cell culture or a three-dimensional (3D) cell culture comprising differentiated embryonic stem cells or induced pluripotent stem cells.

16. The method of claim 14, wherein the first performance or the second performance comprises success or failure at completing a reinforcement learning task, a level of competence at the reinforcement learning task, or a speed of learning the reinforcement learning task.

17. The method of claim 14, wherein a reward used to train the neuronal cell culture to perform the first reinforcement learning task is the same as the reward used to train the neuronal cell culture to perform the second reinforcement learning task.

18. The method of claim 17, wherein the reward is electrical stimulation, light stimulation, or chemical stimulation applied to the neuronal cell culture.

19. The method of claim 14, further comprising generating a comparison of the first performance of the neuronal cell culture on the first reinforcement learning task to the second performance of the neuronal cell culture on the second reinforcement learning task.

20. The method of claim 14, further comprising:
training the neuronal cell culture to perform a third reinforcement learning task by communicating with the neuronal cell culture using the standard protocol for providing inputs and receiving outputs via the interface connected to the electronic computing device; and
recording a third performance of the neuronal cell culture on the third reinforcement learning task.
